(19) United States
(12) Patent Application Publication
(10) Pub. No.: US 2009/0205664 A1
(43) Pub. Date: Aug. 20, 2009

(54) ULTRA-VIOLET GERMICIDAL PERSONAL PROTECTION APPARATUS

(75) Inventor: Donald E. Lyon, Hanwell (CA)

Correspondence Address: SMART & BIGGAR, 438 UNIVERSITY AVENUE, SUITE 1500, BOX 111, TORONTO, ON, M5G 2K8, CA

(73) Assignee: UV LIGHT SCIENCES GROUP, INC., St. Stephen, NB (CA)

(21) Appl. No.: 12/092,666

(22) PCT Filed: Mar. 20, 2006
(86) PCT No.: PCT/CA2006/000396

§ 371 Date: May 5, 2008

(30) Foreign Application Priority Data
Nov. 3, 2005 (CA) PCT/CA2005/001688

(51) Int. Cl. B01D 53/00; A61M 16/06

(52) U.S. Cl. 128/205.12; 422/168; 422/105

(57) ABSTRACT

A practical, germicidal, personal protection system may be worn by a user to kill or deactivate germs, viruses or other pathogens, which are located in the air to be breathed by the user. Before entering a mask, a hood or a suit worn by the user, air is exposed, in a sterilization unit, to Ultra-Violet C-band (UVC) radiation. Advantageously, the UVC radiation is lethal to undesirable germs, viruses and other pathogens. In this manner, pathogen-free air may be provided to the user. Bulbs used to generate UVC radiation are known to also promote the creation of ozone. Accordingly, the personal protection system includes means to minimize the ozone in the air that ultimately reaches the user. A similar personal protection system may also be used expose, to UVC radiation, breath exhaled from the user, thereby killing any germs, viruses or other pathogens exhaled by the user.

### FIELD OF THE INVENTION

The present invention relates to apparatus for prevention of transmission of germs and other pathogens from an ambient environment to the user of the apparatus. More specifically, the present invention relates to a germicidal, personal protection apparatus in which air input to the apparatus is subjected to Ultra-Violet (“UV”) radiation.

### BACKGROUND

UV radiation in a wavelength range of 250-270 nm may be effectively used to sterilize air by killing or deactivating pathogens in the air. The effectiveness of sterilization is related to UVC exposure level, which is dependant on the duration of exposure and intensity of the UVC radiation. The duration of exposure depends on the rate of air flow through the sterilization unit.

A UV germicidal respirator system is disclosed in U.S. Pat. No. 5,165,395, issued Nov. 24, 1992 to Ricci (hereinafter “Ricci”) and currently owned by the applicant. Ricci discloses a UVC protection mask system using a miniature ultra-violet lamp as a UVC radiation source. However, the intensity of UVC radiation produced by standard miniature ultra-violet lamps may be insufficient in some circumstances, particularly where a high rate of air flow in the respirator is required.

There remains a need for a practical, effective UV germicidal respirator system adapted to provide sufficient UV radiation sterilization of air to safely and effectively protect users.

### SUMMARY

Commercially available UVC bulbs may produce a sufficient intensity of UVC radiation to effectively sterilize air at a flow rate through a sterilization chamber sufficient for normal user respiration. The present invention provides for the design of novel, compact air sterilization respiration systems that use such bulbs.

Generally, required sterilization levels will vary depending upon the application to which the air sterilization respiration system is to be used and the types of pathogens that must be killed or deactivated. Further, as noted, the effectiveness of sterilization is related to UVC exposure level, which is dependant on the duration of exposure and intensity of the UVC radiation. Appropriately sized UVC bulbs are accordingly provided in sterilization chambers having dimensions and characteristic adapted to subject the air being treated to exposure for the required duration to the intensity of UVC radiation. Further still, the effectiveness of sterilization may be enhanced by exposure of the air to ozone which may be produced as a result of generation of UVC radiation using suitable bulbs. However, ozone should be removed from the sterilized air before being inhaled by the user and the present invention accordingly provides for the use of ozone creating bulbs in combination with ozone removal means. Removal of ozone and use of appropriate filters increases the resistance of the system to airflow and generally relatively high air flow must be maintained for efficient working of the system. Accordingly, a fan may be provided to ensure adequate airflow. Reliability of the system and safety of the user must also be taken into account and the present invention provides means to minimize the likelihood of bulb breakage and means for monitoring and controlling the radiation intensity and air flow rate.

Accordingly, the present invention provides a practical, germicidal, personal protection system that includes a sterilization chamber having a sufficiently large UV radiation generating radiation source to effectively sterilize air passing into and/or out of the apparatus, and an ozone removal chamber adapted to remove ozone from treated air before it reaches a user.

In accordance with an aspect of the present invention there is provided an apparatus including a sterilization chamber defining an air flow path from an inlet passageway to an outlet passageway, a radiation source positioned within the sterilization chamber between the inlet passageway and the outlet passageway, the radiation source generating Ultra-Violet radiation in a wavelength range of 250-270 nanometers, a source of electrical power for the radiation source, an ozone removal chamber in fluid communication with the outlet passageway and a means, in the ozone removal chamber, for removing ozone in air output from the sterilization chamber.

In accordance with another aspect of the present invention there is provided an Ultra-Violet germicidal mask system. The Ultra-Violet germicidal mask system includes a mask, a sterilization unit and an air hose connecting an outlet of the sterilization unit to an inlet of the mask. The sterilization unit includes a sterilization chamber defining an air flow path from an inlet passageway to an outlet passageway, a radiation source positioned within the sterilization chamber between the inlet passageway and the outlet passageway, the radiation source generating Ultra-Violet radiation in a wavelength range of 250-270 nanometers, a powered blower for drawing input air into the sterilization unit and compelling a flow of the input air past the radiation source and a source of electrical power for the radiation source and the blower.

In accordance with a further aspect of the present invention there is provided an Ultra-Violet germicidal mask system. The Ultra-Violet germicidal mask system includes a mask, a sterilization unit and an air hose connecting an outlet of the sterilization unit to an inlet of the mask. The sterilization unit includes a sterilization chamber defining an air flow path from an inlet passageway to an outlet passageway, a reflective interior surface of the sterilization chamber adapted to reflect Ultra-Violet radiation, a radiation source positioned within the sterilization chamber between the inlet passageway and the outlet passageway, the radiation source generating Ultra-Violet radiation in a wavelength range of 250-270 nanometers and a source of electrical power for the radiation source.

In accordance with a still further aspect of the present invention there is provided an Ultra-Violet germicidal mask system. The Ultra-Violet germicidal mask system includes a mask, a sterilization unit and an air hose connecting an outlet of the sterilization unit to an inlet of the mask. The sterilization unit includes a sterilization chamber defining an air flow path from an inlet passageway to an outlet passageway, a radiation source positioned within the sterilization chamber between the inlet passageway and the outlet passageway, the radiation source generating Ultra-Violet radiation in a wavelength range of 250-270 nanometers, a vibration isolating mount for maintaining the radiation source in a position spaced from an interior surface of the sterilization chamber and a source of electrical power for the radiation source.

In accordance with still another aspect of the present invention there is provided an Ultra-Violet germicidal mask system. The Ultra-Violet germicidal mask system includes a mask, a sterilization unit and an air hose connecting an outlet of the sterilization unit to an inlet of the mask. The sterilization unit includes a sterilization chamber defining an air flow path from an inlet passageway to an outlet passageway, a radiation source positioned within the sterilization chamber between the inlet passageway and the outlet passageway, the radiation source generating Ultra-Violet radiation in a wavelength range of 250-270 nanometers, a thin film coating on the radiation source, the coating having a characteristic destructive interference pattern for electromagnetic radiation with a wavelength between 185-187 nm and a characteristic constructive interference pattern for electromagnetic radiation with a wavelength between 250-270 nm and a source of electrical power for the radiation source.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures which illustrate example embodiments of this invention:

FIG. 1 illustrates a germicidal mask system including a sterilization unit in accordance with the present invention;

FIG. 2 is a front perspective view of a first exemplary sterilization unit of the germicidal mask system of FIG. 1;

FIG. 3 is an exploded view of an air pathway in the sterilization unit of FIG. 2;

FIG. 4 is a partially exploded view of the sterilization unit of FIG. 2 including the air pathway of FIG. 3;

FIG. 5 is a cross section of the sterilization unit of FIG. 2 along the line shown in FIG. 4;

FIG. 6 is an exploded rear view of the sterilization unit of FIG. 2 including the air pathway of FIG. 3;

FIG. 7 is an end view of a sterilization chamber body portion of the air pathway of FIG. 3;

FIG. 8 illustrates a UV bulb mounting mechanism that provides shock and vibration isolation in accordance with an embodiment of the present invention;

FIG. 9 is a front perspective view of a second exemplary sterilization unit for use in the germicidal mask system of FIG. 1;

FIG. 10 is a top view of the sterilization unit of FIG. 9 with a top housing removed;

FIG. 11 is a rear view of the second exemplary sterilization unit of FIG. 9;

FIG. 12 is a bottom view of the second exemplary sterilization unit of FIG. 9 with a bottom housing and catalyst chambers removed;

FIG. 13 is a bottom plan view of the second exemplary sterilization unit of FIG. 12 with a manifold removed;

FIG. 14 is a sectional view of the exemplary sterilization unit of FIG. 9 as shown in FIG. 10;

FIG. 15 schematically illustrates a third exemplary sterilization unit according the present invention; and

FIG. 16 schematically illustrates a fourth exemplary sterilization unit according to the present invention.

### DETAILED DESCRIPTION

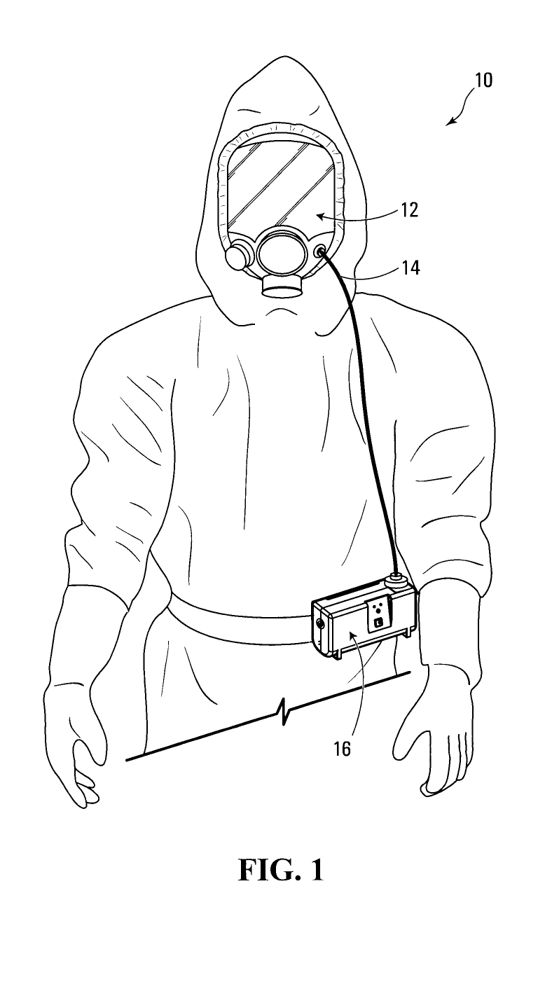

In a first embodiment exemplary of the present invention, a UV germicidal respirator system 10 includes a face mask 12 and a sterilization unit 16 as shown in FIG. 1. The non-porous face mask 12 may be flexible and form a generally airtight fit over the face of the user, including the nose and mouth. The face mask 12 may, for instance, be a mask certified by the US National Institute for Occupational Safety and Health (NIOSH). The sterilization unit 16 is in fluid communication with the non-porous face mask 12 by way of an air hose 14.

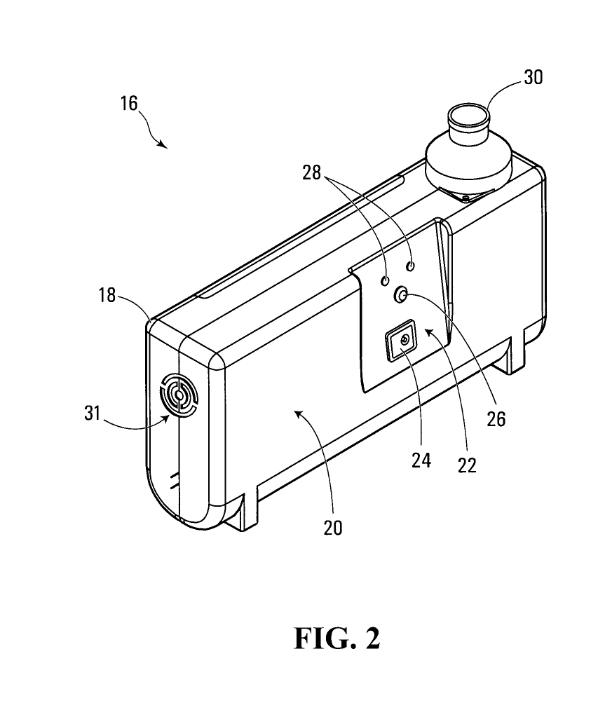

FIGS. 2-6 illustrate a first embodiment of a sterilization unit 16 in accordance with the invention. The unit 16 includes a back housing 18 and a front housing 20, formed in a conventional manner of plastic.

The front housing 20 is indented to form a control plate portion 22 that includes an aperture in which a grommet 24 is mounted, an aperture through which an activation switch 26 is accessed and apertures in which light pipes 28 are mounted. An outlet jacket 30 straddles a joint between the back housing 18 and the front housing 20 and provides an output portal for sterilized air and a connection for the air hose 14. The back housing 18 and the front housing 20 are formed so as to define an input portal for input of air. As illustrated in FIG. 2, the input portal may, for example, take the form of input apertures 31.

The jacket 30 may be sized to accommodate connection to standard breathing components including, for example: NIOSH-approved masks; NIOSH-approved hoods; NIOSH-approved suits; anesthesia masks; re-breather masks; oxygen masks; and other types of medical masks.

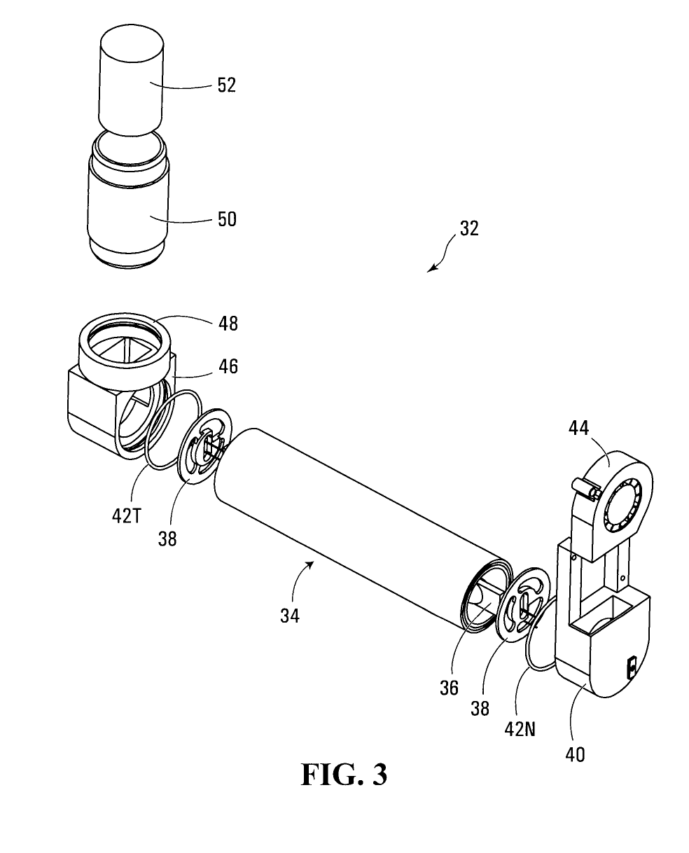

The air pathway 32 of the sterilization unit 16 is illustrated in an exploded view in FIG. 3. The pathway 32 includes an open ended, cylindrical sterilization chamber body 34 formed of a material that is opaque to UV radiation. While the sterilization chamber body 34 in the exemplary embodiment is cylindrical, it will be understood by those skilled in the art that it may have many other configurations.

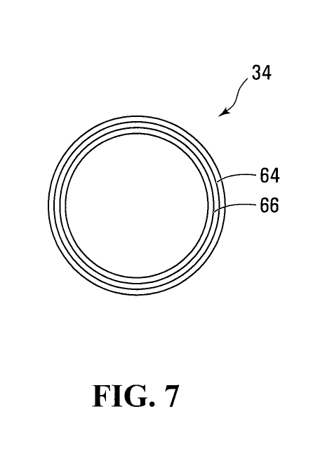

Highly reflective material may be used on the interior of the sterilization chamber body 34 to reflect germicidal UVC radiation in the chamber in order to reduce the required size of the UV bulb 36. Polished aluminum, for instance, may be a suitable highly reflective material for the sterilization chamber body 34. Alternatively, a layer of reflective material 64 (see FIG. 7) may be used on the interior surface of the sterilization chamber body 34. The reflective material 64 may, for instance, be a sintered flouropolymer, such as Spectralon™ by Labsphere® of North Sutton, N.H. Further alternatives for the reflective material 64 may be a thin-foil sintered flouropolymer with an aluminum backing, or barium sulfate paint on a suitable backing substrate. To protect the reflective material 64, a sleeve 66 of highly UVC transmissive material, such as GE 214 glass, may be installed in the interior of the sterilization chamber body 34. As a further alternative barium sulfate paint may be applied to the exterior surface of the sleeve 66 before installation into the sterilization chamber body 34.

A UV bulb 36 is mounted in the sterilization chamber body 34 on bulb mounting plates 38 located at each end of the sterilization chamber body 34. The UV bulb 36 is suspended within the sterilization chamber with leads at each end of the UV bulb 36 extending through and held in apertures in the bulb mounting plates 38.

Many UV radiation sources are available for use as the UV bulb 36. Prior to the present invention, sterilization of air has generally been accomplished with UV radiation sources designed to minimize the amount of ozone produced such as UVC radiation sources using doped quartz glass. In particular, ozone-producing bulbs have not been considered for personal germicidal mask systems because the ozone volume produced by ozone-producing bulbs in closed-in systems may be toxic to the user.

However, minimizing the production of ozone typically reduces the germicidal efficiency of the UV radiation source. Air sterilization systems that satisfy current NIOSH flow requirements may be required to sterilize air flowing at 170 liters/minute or, in some cases, greater flow rates. In order to sterilize air at such a high flow rate and maintain a compact sterilization unit, UVC intensity in the sterilization chamber may be increased through the use of ozone-producing bulbs. For instance, non-doped quartz glass bulbs yield UVC output transmission characteristics with UVC intensity that is comparatively higher than corresponding doped bulbs because doping not only leads to absorption of ozone producing radiation but also typically absorbs useful germicidal UVC radiation.

In accordance with one aspect of the present invention, it is proposed herein to employ an ozone-producing UV radiation source as the UV bulb 36 to achieve efficient sterilization within the sterilization chamber body 34 by increasing the relative intensity of the radiation and due to the fact that ozone is known to have germicidal properties. As discussed below, means are provided to minimize the concentration of ozone in the sterilized air before it reaches the user.

The bulb size and type required will depend upon many factors. One factor is the UV exposure level required to destroy or deactivate the pathogens of interest. For example, destruction of typical influenza requires 6,500 μJ/cm² and 10,500 μJ/cm² is required for Tuberculosis. It has been found that around 15,000 μJ/cm² is enough to kill most common pathogenic material. Another factor is the required flow rate of the output of sterilized air. Air sterilization systems that satisfy current NIOSH flow requirements may be required to sterilize air flowing at 170 liters/minute. Furthermore, the configuration of the sterilization unit should be taken into account. In particular, variables such as the length and cross-sectional area of the sterilization chamber, the degree of reflection available from the interior surface of the sterilization chamber and the distance between the UV bulb and the interior of the sterilization chamber. The Applicants have successfully employed a 10 W T5 format bulb, where T5 refers to a tubular bulb with a ⅝ inch (16 mm) diameter.

Notably, a longer bulb that is rated to provide the same degree of UV exposure as a smaller bulb may be preferred, as a given unit of pathogenic material will be subjected to the UV exposure for a longer duration (for the same flow rate).

The UV bulb 36 may be oversized in wattage to allow for the characteristic reduction in intensity that occurs over the life of the UV bulb 36 and thus ensure sufficient intensity exists for the desired UVC exposure level. Most fluorescent mercury vapor bulbs have a characteristic lifetime reduction in intensity of around 20%.

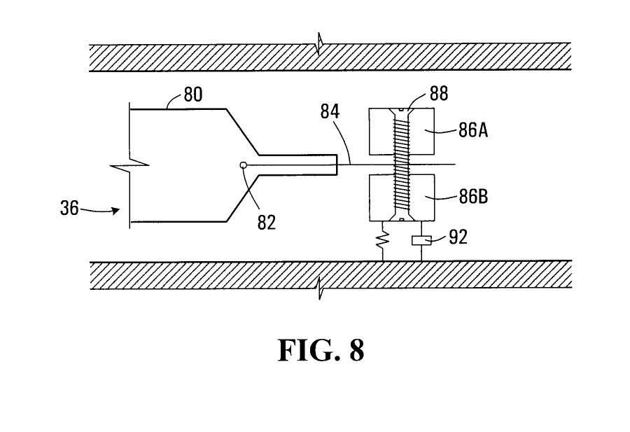

Since shattered mercury vapor bulbs release ionized mercury gas, if such bulbs are used, vibration and shock protection may be advisable. An alternative embodiment of the sterilization chamber adapted for such protection is disclosed in FIG. 8. A standard UV bulb 36 having a filament 82 at each end of a quartz glass encasement 80 and strong metal leads 84 extending from the filament 82 out of the quartz glass encasement 80 is suspended in the sterilization chamber body 34 by engagement of the leads 84 in clamps mounted in the sterilization chamber body 34. For instance, a clamp may be mounted toward each end of the sterilization chamber body 34, with each clamp having an upper jaw 86A and a lower jaw 86B. A tightening mechanism 88 is provided to adjust the distance between the upper jaw 86A and the lower jaw 86B to provide for secure mounting and ease of replacement of the bulb as required. The lower jaw 86B is connected to the sterilization chamber 34 through a damping mechanism 92. The leads 84 at each end of the UV bulb 36 are engaged by the clamps thereby providing vibration isolation between the UV bulb 36 and the sterilization chamber body 34 and significantly reducing the likelihood of the UV bulb 36 breaking due to contact between the UV bulb 36 and the sterilization chamber body 34.

Referring to FIG. 3, an input collar 40 is mounted at an input end of the sterilization chamber body 34 such that the sterilization chamber is in communication with a cavity through the input collar 40. A silicone o-ring 42N forms a seal between the input end of the sterilization chamber body 34 and the input collar 40. A blower 44 may be fastened to the input collar 40 such that output from the blower 44 is received by the cavity in the input collar 40 and, subsequently, the sterilization chamber. The blower 44 is used to assist in providing required air flow through the sterilization unit 16 and to cool the UV bulb 36.

An elbow joint 46 is mounted at an output end of the sterilization chamber body 34 such that the sterilization chamber is in communication with a cavity through the elbow joint 46. A silicone o-ring 42T forms a seal between the output end of the sterilization chamber body 34 and the elbow joint 46. The elbow joint 46 includes an output collar 48 adapted to receive the bottom end of an ozone removal chamber body 50 defining an ozone removal chamber. Output air from the sterilization chamber body 34 passes through the cavity in the elbow joint 46 and subsequently through an ozone removal means, in the illustrated embodiment, ozone removal chamber 50.

The ozone removal means may use any of a number of suitable known methods. As discussed further in describing the first illustrated embodiment, one option is to use a catalyst system to convert ozone to normal (diatomic) oxygen. A second option is to use an ozone-absorption filter, such as an activated carbon filter, to absorb ozone. A third option is to use a coating applied to the bulb to reduce the emission of radiation with a wavelength less than 242 nm (which is known to lead to ozone production). Appropriately selected and deposited materials are known to sharply diminish ozone production as a by product of UV radiation propagation to the point that neither catalytic ozone destruction nor ozone-absorption filtering is required. For instance, a suitable coating material will have a characteristic destructive interference pattern for electromagnetic radiation with a wavelength between 185-187 nm and a characteristic constructive interference pattern for electromagnetic radiation with a wavelength between 250-270 nm to allow better than 85% transmission of radiation from the interior of the UV bulb 36. Such material may be deposited on the bulb as a thin film, for instance, by the known sol-gel process, sputtering or by vapor deposition.

An advantage of coating the UV bulb 36 is that the production of ozone is significantly reduced. However, while ozone is toxic to the user, ozone is also toxic to pathogenic material. Accordingly, as long as the ozone can be safely handled, then allowing the production of ozone maximizes the germicidal effect per watt of electrical power used by the sterilization unit 16.

If the UVC radiation level requirements of the system allow, a conventional ozone-minimizing UV radiation source to be used for the UV bulb 36. Unfortunately, such minimizing UV radiation sources still produce ozone at levels that may not be safe for closed-in personal protection systems of the type disclosed herein. Accordingly, steps should still be taken to reduce ozone in the air reaching the user, such as coating the radiation source to further reduce sub-242 nm radiation emission, or use of an ozone-destruction catalyst or ozone-absorption filter.

In the embodiment illustrated in FIG. 3, an ozone destruction catalyst 52 is installed in the ozone removal chamber. Suitable materials for the catalyst 52 include Carulite® 200, a manganese dioxide compound from the Carus Chemical Co. of Peru, Ill. and PremAir® by Engelhard Corporation of Iselin, New Jersey. Notably, typical ozone-destruction catalyst systems require a sufficient residence time for the necessary chemical reaction. In addition, many ozone-destruction catalyst systems have a minimum airflow velocity requirement to work efficiently. The dimensions of the ozone removal chamber body 50 are determined taking into account residence time and airflow velocity requirements.

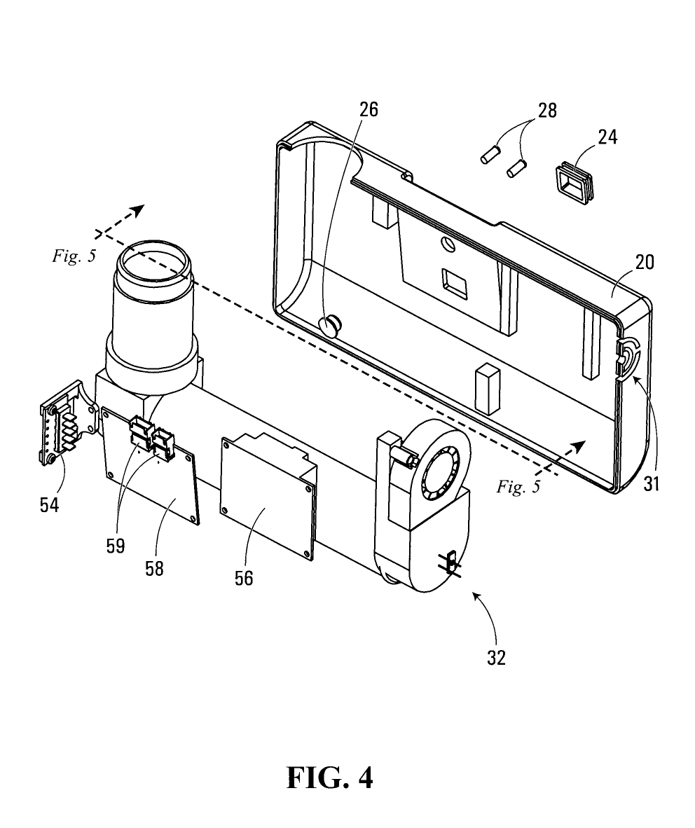

FIG. 4 illustrates the arrangement of battery connector board assembly 54, lamp ballast module 56, control circuit board 58 and activation switch 26 in relation to the air pathway 32 and the front housing 20. The control circuit board 58 includes sockets 59 for mounting light emitting diodes (LEDs). When the LEDs (not shown) are installed in the control circuit board 58 and the control circuit board 58 is installed in the front housing 20, the LEDs are positioned to emit light through the light pipes 28 to the exterior of the front housing 20. The control circuit board 58 may include an input receptacle for receiving an end of a power cable, access to which is provided through the aperture in the control plate portion 22 that is sealed with the grommet 24.

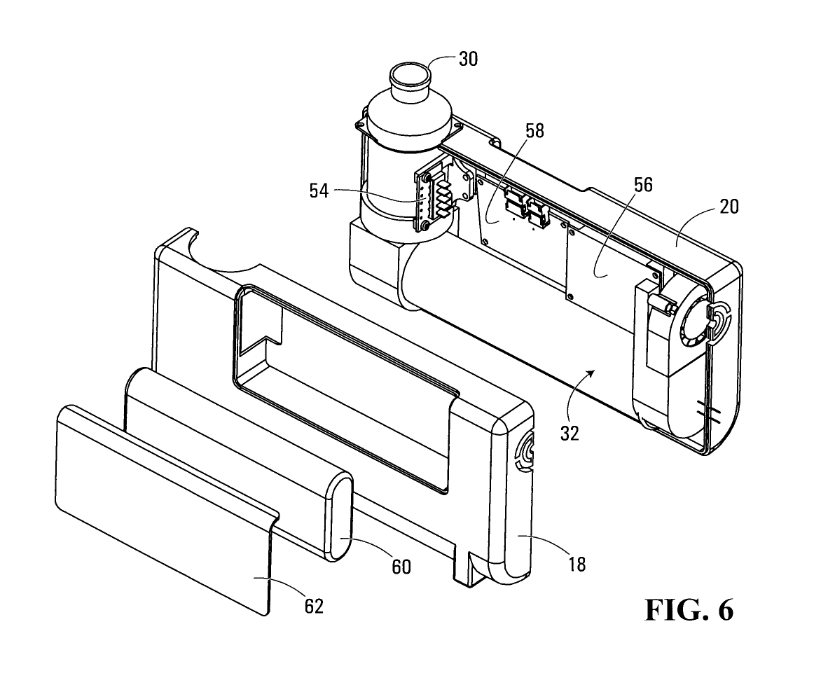

FIG. 6 shows the air pathway 32, the battery connector board assembly 54, the lamp ballast module 56 and the control circuit board 58 installed in the front housing 20. The position of the input apertures 31 correspond to the position of the input to the blower 44. The back housing 18, a battery 60 and a battery cover 62 are also illustrated in FIG. 6. The battery cover 62 engages the back housing 18 in a snap fit to cover the aperture into which the battery 60 fits.

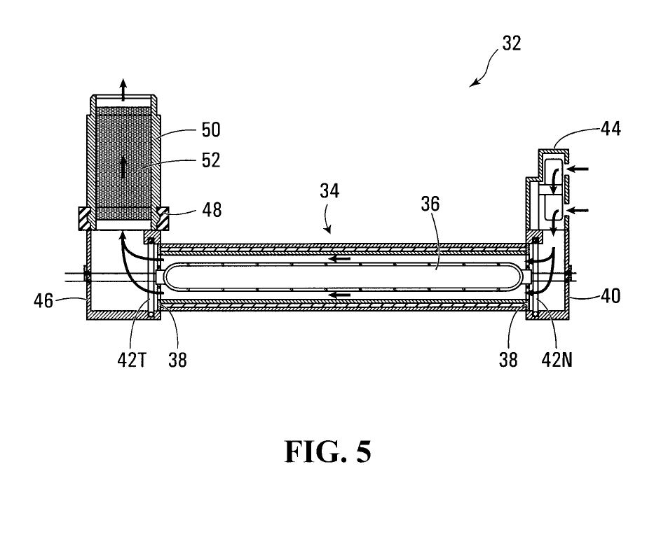

Operation of the sterilization unit 16 may be considered while reviewing FIG. 5. The user activates the sterilization unit 16, through use of the activation switch 26 (see FIG. 2). Input air, which potentially carries pathogens, is drawn into the sterilization unit 16, by the blower 44, through the input apertures 31. The input air is propelled, typically at a rate of between 60 and 300 liters/minute, by the blower 44, into the cavity in the input collar 40. The input air then passes into the sterilization chamber where it is subjected to UVC radiation from the UV bulb 36 and pathogens present in the input air are killed or deactivated. The treated air then passes through the cavity in the elbow joint 46 into the ozone removal chamber and through the catalyst 52. Upon leaving the ozone removal chamber body 50, the treated air passes out of the sterilization unit through the outlet jacket 30, into the input end of the air hose 14 (see FIG. 1) and then into the non-porous face mask 12 to be inhaled by the user.

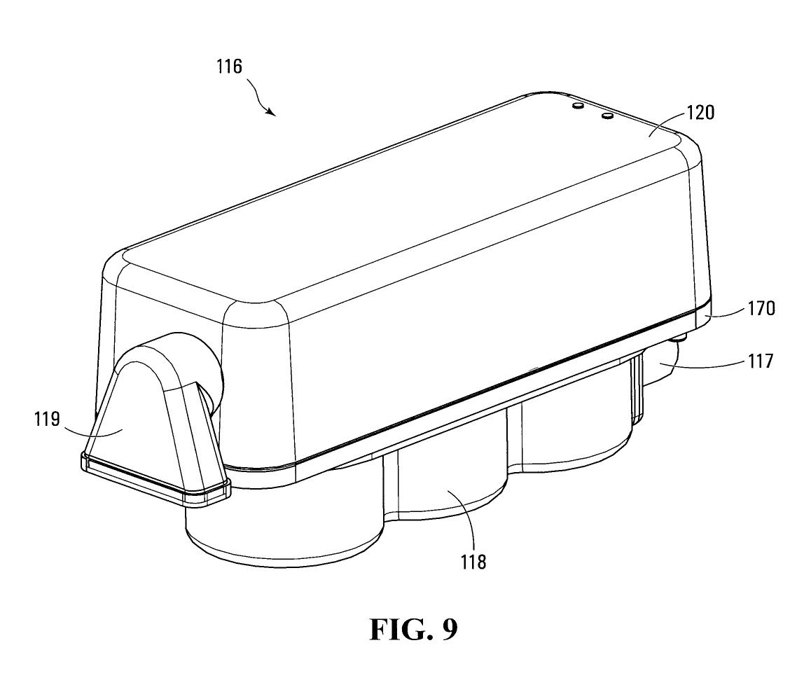

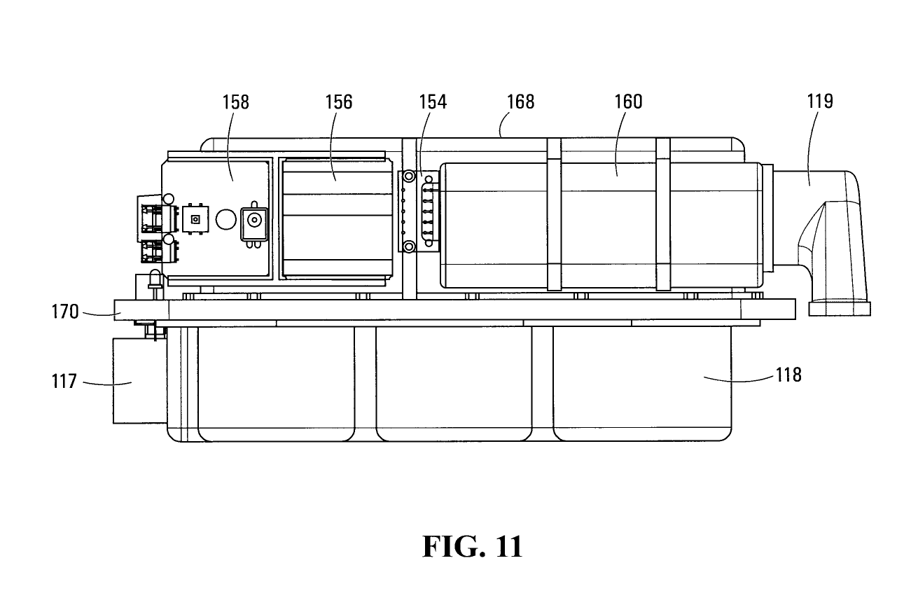

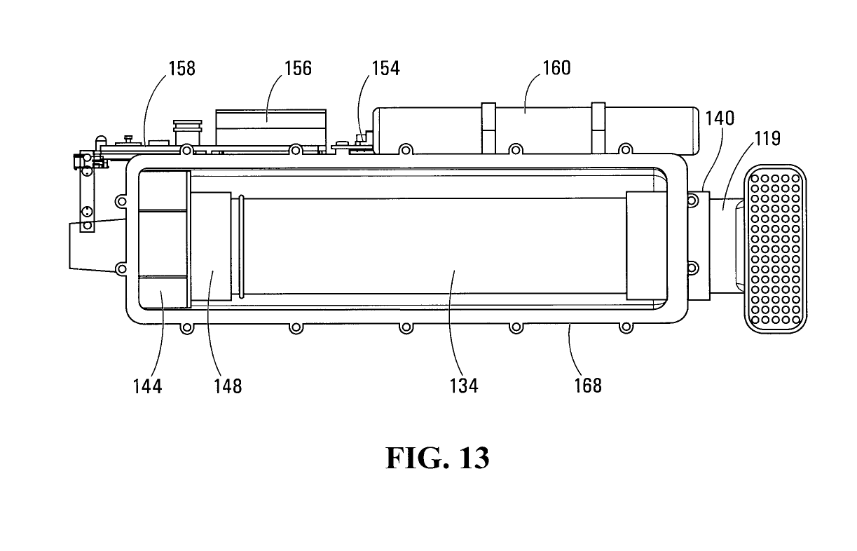

Referring to FIGS. 9-14 a second exemplary sterilization unit 116 has a manifold 170, a top housing 120 and a bottom housing 118 which may be formed of a plastic opaque to Ultra-Violet (UV) radiation. An input portal 119 extends from the left end of the top housing 120. The shape (90 degree bend) of the input portal 119 is designed to act as a baffle, i.e. to reduce or eliminate the escape of UV radiation from the interior of the second exemplary sterilization unit 116.

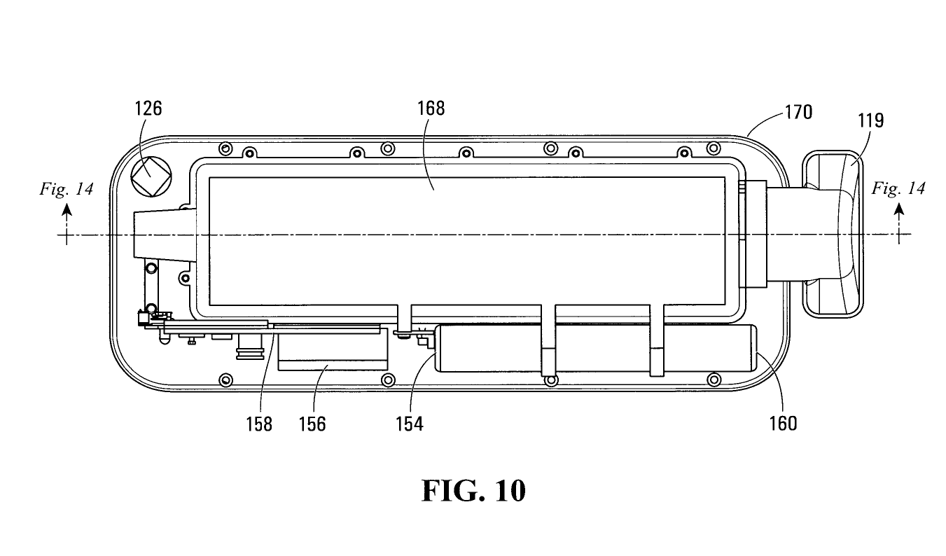

Referring to FIG. 10, showing a top plan view of the second exemplary sterilization unit 116 with the top housing 120 removed, a battery 160 is connected to a battery connector board assembly 154 used to pass electrical energy from the battery 160 to a lamp ballast module 156 and a control circuit board 158. An activation switch 126 is also provided. Tube box 168 is illustrated, attached to the manifold 170, with the input portal 119 extending from the tube box 168.

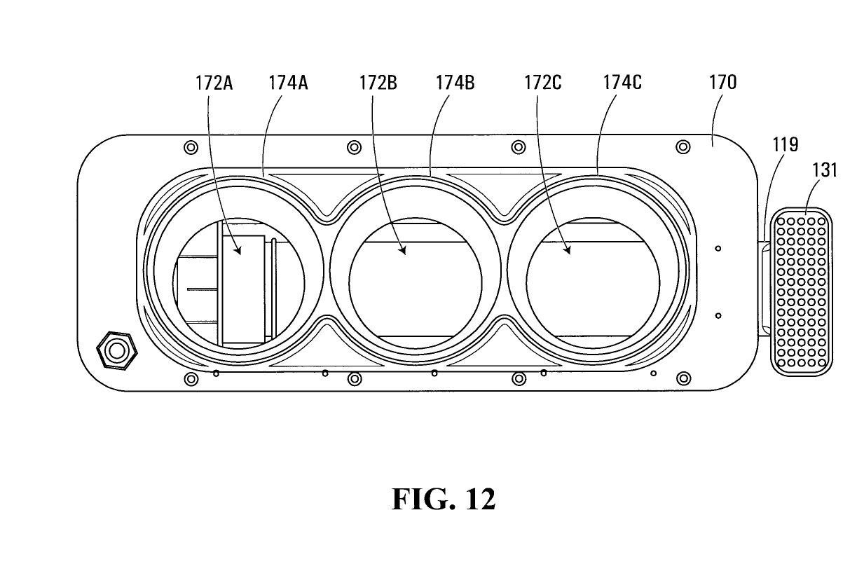

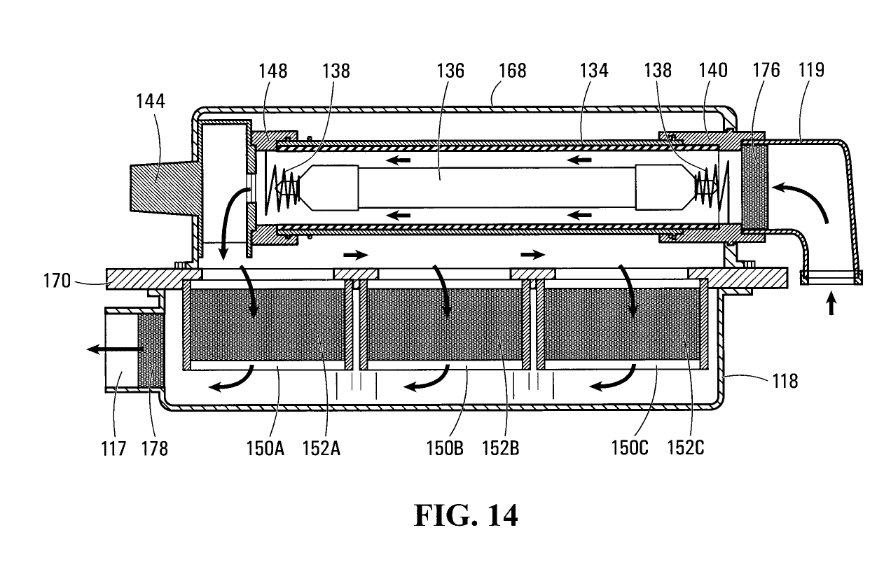

A bottom plan view of the second exemplary sterilization unit 116 is illustrated in FIG. 12 with the bottom housing 118 and catalyst chamber bodies (discussed below) removed. Manifold 170 has three circular apertures 172A, 172B, 172C (individually or collectively 172) and three corresponding threaded cavities 174A, 174B, 174C. The three threaded cavities 174A, 174B, 174C allow the attachment of three catalyst chamber bodies 150A, 150B, 150C (see FIG. 14) to the manifold 170 and apertures 172 allow sterilized air to pass from the interior of the tube box 168 into the ozone removal chamber bodies 150A, 150B, 150C (individually or collectively 150). Catalyst material 152A, 152B, 152C (individually or collectively 152) is installed in the catalyst chamber within each corresponding ozone removal chamber body 150.

Referring to FIG. 14, the tube box 168, moving from right to left, includes an input collar 140 connecting the input portal 119 to a sterilization chamber body 134 and an output collar 148 connecting the sterilization chamber body 134 to a blower 144. A UV bulb 136 is suspended within the sterilization chamber having a pin at each end held fast by a bulb mounting spring 138 at each end of the sterilization chamber body 134. Advantageously, the bulb mounting springs 138 are formed of a conducting material such that they act as part of the circuit providing electrical power from the lamp ballast module 156 to the UV bulb 136. Springs of bare, conducting material are preferable to insulated wires as a means of providing power to the UV bulb 136, since it is known that ozone can be highly corrosive to insulation found on typical insulated wires. Advantageously, the bulb mounting springs 138 also provide shock and vibration protection for the UV bulb 136.

Although a typical low-pressure, instant-start, mercury vapor bulb has two leads on each end, electrical connection is only made to one lead on each bulb end and the current carries through the ionized gas in the bulb. As such, a UV bulb with a strong single pin at each end, as illustrated in FIG. 14, may provide a suitable electrical connection.

The options, discussed above, for reducing the volume of ozone in the treated air are also available for the second exemplary sterilization unit 116, namely: a catalyst system (as shown in FIG. 14); an ozone-absorption filter; and a thin-film coating for the UV bulb 136. Additionally, the interior surface of the sterilization chamber body 134 may be coated with a reflective material and the reflective material may be protected by a sleeve of highly UVC transmissive material as discussed above. It will also be understood that additional features discussed below may also be applied with necessary modifications to the first embodiment discussed above.

Referring again to FIG. 14, an inlet filter 176 may be installed in the input portal 119 to minimize the build-up of dust and dirt on the interior surface of the sterilization chamber. The inlet filter 176 is mounted such that it is readily replaceable as part of a normal system maintenance regimen. Alternatively, the inlet filter may be a replaceable, NIOSH-approved canister (not shown) adapted to prevent the entry of chemicals and/or radiological material into the sterilization chamber body 134. To attach the canister to the sterilization unit 16 would require the replacement of the bent input portal 119 with an input portal that is adapted to provide a standard attachment mechanism for the NIOSH-approved canister. As will also be appreciated by persons of ordinary skill in the art of air sterilization, the use of an inlet filter in the form of a NIOSH-approved canister, in conjunction with use of hardened outer surfaces for the bottom housing 118, the manifold 170 and the top housing 120, may allow the second exemplary sterilization unit 116 to be effective in CBRN (chemical/biological/radiological/nuclear) hazard environments.

As also shown in FIG. 14, an outlet filter 178 may be installed in the outlet portal 117. When manganese dioxide based ozone destruction catalyst materials, are used as the catalyst materials 152, filters suitable for use as the outlet filter 178 should prevent particles greater than 5 microns from passing through. Such filters are adapted to catch manganese-based dust that may be released through the use of such catalyst material 152.

The structure for the outlet filter 178 may, additionally or alternatively, be selected to act as a backup filter to prevent the passage of pathogenic material in the case of failure of the UVC bulb 136. For instance, filters with a NIOSH N95 rating, rated to be at least 95% effective at stopping particles 0.3 microns or larger may be used. The need for such a backup filter will depend upon the application and level of risk. For instance, where size and weight are important and risk is acceptable, no backup outlet filter may be required, but the outlet filter for backup purposes may be considered critical where the germicidal mask system 10 is to be used when, for instance, dealing with highly contagious diseases. In the absence of an outlet filter, or in addition thereto, a user may want to utilize other protective measures, such as wearing a surgical cloth mask under the non-porous face mask 12.

As noted, a blower 144 is provided in sterilization unit 116. The blower overcomes airflow obstructions in the system including any inlet filter 176, catalyst material 152, outlet filter 178 and the drag of the air hose 14. Moreover, powered ventilation may be mandated to satisfy certification requirements for many NIOSH classes of respirator equipment.

Powered ventilation has additional benefits including that increasing airflow past the UV bulb 136 provides a cooling effect. Additional cooling of sterilized air may be effected by installing a suitable electronic heat sink (not shown) in the tube box 168. One example of a suitable electronic heat sink would be a Peltier device such as Type # inbS1-031.015 from INB Products of Van Nuys, Calif.

The blower 144 may be controlled to maintain a minimum flow rate by incorporating an air flow rate sensor (not shown) in the sterilizer chamber body 134. The air flow rate sensor may, for example, be integrated into the design of the blower 144. A blower controller in the control circuit board 158 may be used to monitor and adjust air flow rate by comparing the output of the air flow rate sensor to a desired flow rate and controlling the speed of the blower 144 based on the difference. The blower controller may employ an integration function to eliminate differences/errors in the air flow rate. Such a closed-loop blower control system can compensate for dust collecting in the inlet filter 176, the catalyst material 152 or the outlet filter 178 and other variables that affect air flow in the sterilizer.

A closed-loop blower control system is also important for variants of the germicidal mask system 10, which may be used in medical devices allowing medical personnel to set a specific air flow rate. In such variants, the blower control system is configured to allow air flow rate to be set by medical personnel. A variable air flow rate set-point may be provided for to allow the air flow rate to be set to a primary value for periods of normal respiration (awake user) and a secondary value for periods of low respiration (asleep user), thereby achieving a required UVC exposure level while optimizing use of the battery 160.

To achieve high flow rates in conjunction with high UVC exposure levels, it may be necessary to use multiple air inlet portals, each inlet portal having a corresponding air pathway including a blower, a UVC sterilization chamber and an ozone-destruction catalyst chamber, all connected by appropriate manifolds between the appropriate components.

Operation of the second exemplary sterilization unit 116 may be considered while reviewing FIG. 14. The user activates sterilization unit 116 using activation switch 126. Input air, which potentially carries pathogens, is drawn into the sterilization unit 116, by the blower 144, through the input portal 119 and inlet filter 174. The input air passes into the sterilization chamber body 134. While in the sterilization chamber body 134, the input air is treated to UVC radiation from the UV bulb 136 and pathogens present in the input air are killed or deactivated. The treated air then passes through the blower 144 and into the interior of the tube box 168.

As the tube box 168 forms an airtight seal with the manifold 170, the treated air can only leave the interior of the tube box 168 through the circular apertures 172 in the manifold 170, and thus through the ozone removal chamber bodies 150 and catalyst material 152. Upon leaving the ozone removal chamber bodies 150, the treated air is received in the interior of the bottom housing 118 and then exits through the outlet filter 178 in the outlet portal 117 integral to the bottom housing 118 and then to the user, for instance through a tube, mask, etc.

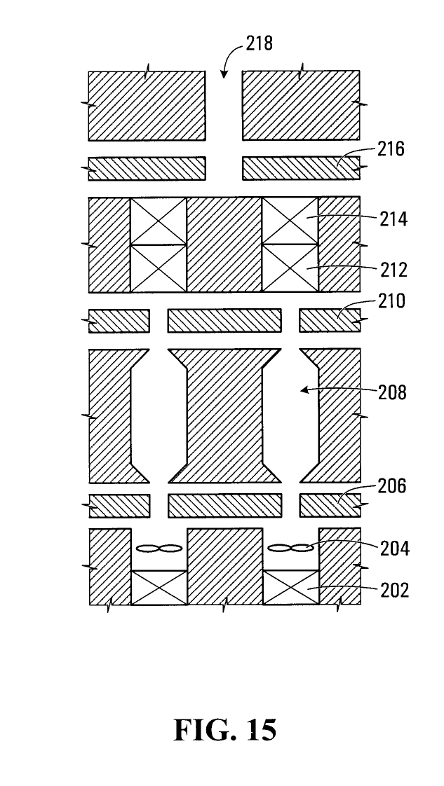

FIG. 15 schematically illustrates a third exemplary sterilization unit. Two inlet portals are illustrated, each inlet portal having an inlet filter 202 and a blower 204. The two inlet portals are in fluid communication with an input air manifold 206, which is in turn in fluid communication with sterilization chambers 208 having UV bulbs installed therein (not shown) corresponding to each of the inlet portals. The sterilization chambers 208 in turn communicate with an ozone catalyst manifold 210, on the opposite side of which is a pair of catalyst chambers, each catalyst chamber housing an ozone catalyst 212 for removing ozone from the treated air and an outlet filter 214 for removing dust output from the catalyst 212. The output of the outlet filters 214 is received by an outlet manifold 216 and, subsequently, an outlet passageway 218.

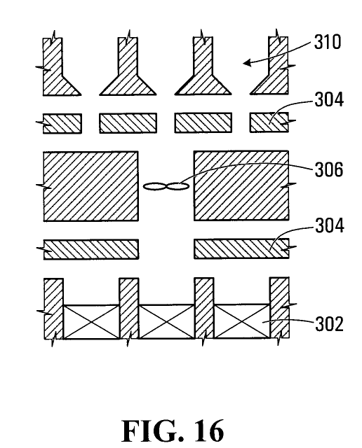

FIG. 16 schematically illustrates the inlet stage of a fourth exemplary sterilization unit. Three inlet portals are illustrated, each inlet portal having an inlet filter 302. The three inlet portals end at an input manifold 304, having a single blower 306. A UV inlet manifold 308 channels the output of the blower 306 into three sterilization chambers 310 having UV bulbs installed therein (not shown).

As will be understood by a person of ordinary skill in the art of air sterilization, although the exemplary sterilization units schematically illustrated in FIGS. 15 and 16 have straight-line air pathways, sterilization units based on such designs may have many three-dimensional direction changes that allow for compactness.

Generally, air exhaled by the user of the germicidal mask system 10 of FIG. 1 will leave the mask 12 by way of a one-way valve. Alternative embodiments of the present invention may provide for sterilization of air exhaled by the user in the same manner as the air inhaled by the user by directing the exhaled air through a sterilization chamber as it exits the system. In an enhanced germicidal mask system in accordance with the invention (not shown), exhaled air sterilization is accomplished through a separate UVC sterilization system that is similar to the inhaled air sterilization but which optionally may include ozone removal means. An air inlet passageway is connected to the one-way exhaust valve of the mask. This passageway connects to its own distinct UVC sterilization chamber for sterilizing the exhaled air, which in turn connects to an air outlet passageway for releasing the exhaled air to the atmosphere.

The germicidal mask system of the present invention is designed to be used more than once and should be configured for decontamination after use. While the face mask 12 (or hood or suit) and air hose 14 may be disposable, the sterilization unit 16 may readily be made reusable. For instance, a lid (not shown) may be provided for covering the open end of the outlet jacket 30. Additionally, a cover (not shown) may be provided for sealing the input apertures 31. Once the inlet and outlet portals are closed, the sterilization unit 16 may be subjected to a decontaminating wet wash.

Alternatively or additionally, the back housing 18 and the front housing 20 may be formed of UVC transparent materials, in which case the entire sterilization unit 16 may be decontaminated using UVC radiation external to the sterilization unit 16. A suitable UVC transparent material for such an application includes a fluoropolymer resin, such as Polyvinylidene fluoride Solef®, Ethylene-chlorotrifluoroethylene Halar® and perfluoroalkoxyl Hyflon®, all from Solvay Solexis of Bollate, Italy.

Other variations related to safe and reliable operation the germicidal mask system 10 include possible modifications to enhance the efficiency of the UV bulb 36 and integration of vibration and shock protection for the UVC bulb 36.

Typical fluorescent mercury vapor bulbs are intended to be part of an alternating current (AC) circuit. The lamp ballast module 56 is provided to convert the DC power supplied by the battery 60 to AC power as required by the UV bulb 36. As is common for portable devices, the battery 60 may be rechargeable. In particular, a power cable (not shown) may have one end plugged into a wall receptacle supplying alternating current (AC) power and another end adapted for plugging into the input receptacle on the control circuit board 58. The control circuit board 58 and the battery connector board assembly 54 may then cooperate to charge the battery 60.

Logic implemented by the control circuit board 58 may allow simultaneous operation of the sterilization unit 16 and recharging of the battery 60. Such logic may also allow for hot-swapping of batteries, i.e., the battery 60 may be replaced while the sterilization unit 16 is operating and receiving power via the power cable. The logic executed by the control circuit board 58 may also allow for the use of multiple batteries, such as on a battery belt.

During operation of the UV bulb 36, a bulb monitor circuit (not shown) may measure voltage being provided by battery 60 and may measure a draw of current by the circuitry of the control circuit board 58 used to operate the UV bulb 36. The battery voltage may be assigned a nominal value so that the measured value may be compared to the nominal value to quickly diagnose a problem, i.e., it may be quickly determined when the battery 60 is not supplying enough voltage to operate the UV bulb 36 to provide a suitable level of radiation. When the absolute voltage difference, between the measured value and the nominal value, exceeds a voltage threshold (i.e., the measured voltage falls outside of a predetermined tolerable band), the bulb monitor may indicate the condition by raising an auditory or visual (e.g., green light, red light) operating condition alarm.

Alternatively or additionally, the current draw by the UV bulb 36 may be assigned a nominal value so that the measured current value may be compared to the nominal value to quickly diagnose a problem, i.e., it may be quickly determined when the UV bulb 36 has burned out. When the absolute current difference, between the measured current value and the nominal value, exceeds a current threshold, the bulb monitor may indicate the condition by raising an auditory or visual operating condition alarm.

Rather than comparing the absolute current difference or the absolute voltage difference to an associated threshold, the measured value may be compared to a lower threshold computed by subtracting a small delta value from the nominal value. If the measured value is lower than the lower threshold, an alarm may be raised. Additionally, the measured value may be compared to an upper threshold computed by adding another (or the same) small delta value to the nominal value. If the measured value is higher than the upper threshold, an alarm may be raised. This will facilitate ensuring operation within a predefined range. Similarly, if either the absolute current difference or the absolute voltage difference exceeds an associated threshold, the bulb monitor may immediately shut down the sterilization unit 16 and indicate to the user that the sterilization operation has failed, perhaps by activating a given one of the LEDs with a predetermined color and raising an auditory alarm.

Even while the values measured by the bulb monitor are close to nominal, the bulb monitor may present the user with auditory or visual (e.g., green light, red light) operating condition alarms. Further condition monitoring, in the form of text-based notification of operating status and alarms, can also be included for mission critical applications where risk associated with failure is considered extremely high.

Note that features such as system monitoring, battery swapping capability and control of powered ventilation may be provided by microprocessor control circuitry, say, in the control circuit board 58.

While the sterilization unit 16 is illustrated as part of the germicidal mask system 10 of FIG. 1, it should be appreciated by a person of ordinary skill in the art of air sterilization that the sterilization unit 16 may be scaled for use in the air passageways of medical devices such as ventilators, respirators and anesthesia equipment. When used in such a manner, the sterilization unit 16 may protect patients from hospital-borne, pan-resistant pathogens, such as pneumonia, that can reside within a medical respiration device.

Notably, it is also contemplated that the sterilization unit 16 may be used in air recirculation and emergency oxygen supply systems in use in the commercial airline industry.

Other modifications will be apparent to those skilled in the art and, therefore, the invention is defined in the claims.

We claim:

1. An apparatus comprising:
a sterilization chamber defining an air flow path from an inlet passageway to an outlet passageway;
a radiation source positioned within said sterilization chamber between said inlet passageway and said outlet passageway, said radiation source generating Ultra-Violet radiation in a wavelength range of 250-270 nanometers;
a source of electrical power for said radiation source;
an ozone removal chamber in fluid communication with said outlet passageway; and
a means, in said ozone removal chamber, for removing ozone in air output from said sterilization chamber.

2. The apparatus of claim 1 wherein said means for removing ozone is a catalyst for converting ozone to diatomic oxygen.

3. The apparatus of claim 1 wherein said means for removing ozone is an ozone-absorption filter.

4. The apparatus of claim 3 wherein said ozone-absorption filter includes activated carbon.

5. An Ultra-Violet germicidal mask system comprising:
a mask;
a sterilization unit according to claim 1; and
an air hose connecting an outlet of said sterilization unit to an inlet of said mask.

6. The apparatus of claim 1 further comprising a control circuit in electrical communication with said radiation source and said a source of electrical power.

7. The apparatus of claim 6 wherein said control circuit is adapted to determine a measure of voltage supplied by said source of electrical power.

8. The apparatus of claim 7 wherein said control circuit is adapted to determine an absolute voltage difference between said measure of voltage and a predetermined voltage value.

9. The apparatus of claim 8 wherein said control circuit is adapted to indicate that said absolute voltage difference exceeds a voltage difference threshold.

10. The apparatus of claim 8 wherein said control circuit is adapted to interrupt supply of power to said source of Ultra Violet radiation responsive to said absolute voltage difference exceeding a voltage difference threshold.

11. The apparatus of claim 7 wherein said control circuit is adapted to indicate that said measure of voltage exceeds an upper voltage threshold.

12. The apparatus of claim 7 wherein said control circuit is adapted to indicate that a lower voltage threshold exceeds said measure of voltage.

13. The apparatus of claim 6 wherein said control circuit is adapted to determine a measure of current drawn by said source of Ultra Violet radiation.

14. The apparatus of claim 13 wherein said control circuit is adapted to determine an absolute current difference between said measure of current drawn and a predetermined current value.

15. The apparatus of claim 14 wherein said control circuit is adapted to indicate that said absolute current difference exceeds a current difference threshold.

16. The apparatus of claim 14 wherein said control circuit is adapted to interrupt supply of power to said source of Ultra Violet radiation responsive to said absolute current difference exceeding a current difference threshold.

17. The apparatus of claim 13 wherein said control circuit is adapted to indicate that said measure of current drawn exceeds an upper current threshold.

18. The apparatus of claim 13 wherein said control circuit is adapted to indicate that a lower current threshold exceeds said measure of current drawn.

19. The apparatus of claim 1 further comprising an input portal in fluid communication with said inlet passageway and ambient air outside said sterilization unit.

20. The apparatus of claim 19 further comprising a dust filter between said input portal and said inlet passageway.

21. The apparatus of claim 19 wherein said input portal is adapted to connect to a standard gas filter canister.

22. The apparatus of claim 19 further comprising an output portal in fluid communication with said ozone removal chamber.

23. The apparatus of claim 22 further comprising a dust filter between said ozone removal chamber and said output portal.

24. The apparatus of claim 23 wherein said dust filter is adapted to filter manganese-based dust.

25. The apparatus of claim 22 wherein said output portal is adapted for connection to standard breathing components.

26. The apparatus of claim 22 further comprising a housing for enclosing elements of said sterilization unit between said inlet portal and said outlet portal.

27. The apparatus of claim 26 wherein said housing is formed of material opaque to said Ultra-Violet radiation in said wavelength range.

28. The apparatus of claim 26 wherein said housing is formed of material transparent to said Ultra-Violet radiation in said wavelength range.

29. An Ultra-Violet germicidal mask system comprising:
a mask;
a sterilization unit including:
a sterilization chamber defining an air flow path from an inlet passageway to an outlet passageway;
a radiation source positioned within said sterilization chamber between said inlet passageway and said outlet passageway, said radiation source generating Ultra-Violet radiation in a wavelength range of 250-270 nanometers;
a powered blower for drawing input air into said sterilization unit and compelling a flow of said input air past said radiation source; and
a source of electrical power for said radiation source and said blower; and
an air hose connecting an outlet of said sterilization unit to an inlet of said mask.

30. The system of claim 29 further comprising an air flow sensor for sensing a rate of said flow of said input air past said radiation source.

31. The system of claim 30 further comprising a blower controller adapted to:
receive an indication of a desired rate of flow;
receive an indication of said rate of said flow from said air flow sensor;
determine a difference between said indication of said rate of said flow and said desired rate of flow; and
control said blower based on said difference.

32. The system of claim 29 further comprising an electronic heat sink for cooling air output from said sterilization chamber.

33. An Ultra-Violet germicidal mask system comprising:
a mask;
a sterilization unit including:
a sterilization chamber defining an air flow path from an inlet passageway to an outlet passageway, a reflective interior surface of said sterilization chamber adapted to reflect Ultra-Violet radiation;
a radiation source positioned within said sterilization chamber between said inlet passageway and said outlet passageway, said radiation source generating Ultra-Violet radiation in a wavelength range of 250-270 nanometers; and
a source of electrical power for said radiation source; and
an air hose connecting an outlet of said sterilization unit to an inlet of said mask.

34. The system of claim 33 further comprising:
an Ultra-Violet radiation transmissive lining within said sterilization chamber; and
where said reflective interior surface is a coating between said body and said lining.

35. The system of claim 34 wherein said coating is sintered flouropolymers.

36. The sterilization unit of claim 34 wherein said coating is thin-foil sintered flouropolymers on an aluminum backing.

37. The sterilization unit of claim 34 wherein said coating is barium sulfate paint on a backing substrate.

38. The sterilization unit of claim 34 wherein said coating is barium sulfate paint on an external surface of said Ultra Violet radiation transmissive lining.

39. The sterilization unit of claim 34 wherein said Ultra Violet radiation transmissive lining is formed of glass or plastic.

40. An Ultra-Violet germicidal mask system comprising:
a mask;
a sterilization unit including:
a sterilization chamber defining an air flow path from an inlet passageway to an outlet passageway;
a radiation source positioned within said sterilization chamber between said inlet passageway and said outlet passageway, said radiation source generating Ultra-Violet radiation in a wavelength range of 250-270 nanometers;
a vibration isolating mount for maintaining said radiation source in a position spaced from an interior surface of said sterilization chamber; and
a source of electrical power for said radiation source; and
an air hose connecting an outlet of said sterilization unit to an inlet of said mask.

41. The system of claim 40 further comprising a clamp for clamping a lead of said radiation source and attaching to said vibration isolating mount.

42. The system of claim 40 wherein said vibration isolating mount comprises a spring.

43. The system of claim 42 wherein said spring is electrically conductive for conducting current from said source of electrical power to said radiation source.

44. An Ultra-Violet germicidal mask system comprising:
a mask;
a sterilization unit including:
a sterilization chamber defining an air flow path from an inlet passageway to an outlet passageway;
a radiation source positioned within said sterilization chamber between said inlet passageway and said outlet passageway, said radiation source generating Ultra-Violet radiation in a wavelength range of 250-270 nanometers;
a thin film coating on said radiation source, said coating having a characteristic destructive interference pattern for electromagnetic radiation with a wavelength between 185-187 nm and a characteristic constructive interference pattern for electromagnetic radiation with a wavelength between 250-270 nm; and
a source of electrical power for said radiation source; and
an air hose connecting an outlet of said sterilization unit to an inlet of said mask.
